(19) United States
(12) Patent Application Publication
(10) Pub. No.: US 2017/0336420 A1
(43) Pub. Date: Nov. 23, 2017

(54) METHODS AND KITS FOR TREATING CARDIOVASCULAR DISEASES

(71) Applicant: RAPPAPORT FAMILY INSTITUTE FOR RESEARCH IN THE MEDICAL SCIENCES, Hafia (IL)

(72) Inventors: Andrew LEVY, Haifa (IL); Shany BLUM, Haifa (IL)

(21) Appl. No.: 15/524,054

(22) PCT Filed: Nov. 4, 2015
(86) PCT No.: PCT/IL2015/051067

§ 371 Date: May 3, 2017

Related U.S. Application Data
(60) Provisional application No. 62/074,723, filed on Nov. 4, 2014.

(51) Int. Cl. G01N 33/68; A61K 31/355; A61K 31/455

(52) U.S. Cl. CPC G01N 33/6893; A61K 31/455; A61K 31/355; G01N 2800/32; G01N 2333/4713

(57) ABSTRACT

The present invention is directed to methods and compositions of treating cardiovascular disease or disorder in a subject in need thereof, comprising steps of determining and identifying the haptoglobin phenotype of the subject and thereby selecting the course of treatment with an agent capable of raising HDL.

### FIELD OF THE INVENTION

The invention is directed to methods and kits for treating cardiovascular diseases or disorders in a subject in need thereof, comprising the step of identifying the haptoglobin phenotype of the subject and thereafter selecting a course of treatment.

### BACKGROUND OF THE INVENTION

Cardiovascular diseases (CVDs), are one of the leading causes of death worldwide. CVDs generally refer to conditions that involve narrowed or blocked blood vessels that can lead to a heart attack, chest pain (angina) or stroke. Other conditions, such as infections and conditions that affect the heart's muscle, valves or beating rhythm, are also considered as forms of CVD. The causes of CVD are diverse with atherosclerosis and/or hypertension being the most common. Although cardiovascular disease usually affects old adults, the antecedents of cardiovascular diseases, notably atherosclerosis, begin in early life, making primary prevention efforts necessary from childhood.

Treatment of CVD varies and may include life style changes, medications and medical procedures or surgery. Common medications used to treat cardiovascular diseases include medications to lower the blood pressure, (e.g., diuretics, angiotensin-converting enzyme (ACE) inhibitors and beta blockers), blood thinning medications (e.g., aspirin) or cholesterol-lowering medications (e.g., statins and fibrates). Common medical procedures or surgeries include coronary angiography, percutaneous coronary interventions (PCIs), coronary artery bypass grafting (CABG), and carotid endarterectomy.

In the past few decades various agents aimed at increasing the levels of high density lipoprotein (HDL) in the blood have been used and tested for treating CVD. Those agents include niacin or analogs thereof and a class of drugs that inhibit cholesterylester transfer protein (CETP), termed CETP inhibitors. The mechanism of action of CETP inhibitors involves inhibition of cholesterylester transfer protein (CETP), the protein responsible of transforming HDL to very low density or low density lipoproteins (VLDL or LDL). Both niacin and CETP inhibitors efficiently increase HDL and lower LDL levels in the blood. Thus, those agents were initially believed to reduce the risk of atherosclerosis by improving blood lipid levels. However, despite a demonstrated increase in the HDL blood levels of patients, at least two of the CETP inhibitors (e.g., Torcetrapib®, and Dalcetrapib®) and two of the niacin compositions (e.g., Niaspan® and Tredaptive®) failed in clinical trials, as a result of either causing a marked increase in deaths (Torcetrapib®), or for failing to show clinically meaningful efficacy (e.g., Dalcetrapib® and Niaspan®).

Haptoglobin (Hp) is a hemoglobin-binding serum protein which plays a major role in the protection against heme-driven oxidative stress. In humans, a common polymorphism of the haptoglobin gene, characterized by alleles Hp 1 and Hp 2, gives rise to structurally and functionally distinct haptoglobin protein phenotypes, known as Hp 1-1, Hp 2-1, and Hp 2-2. This polymorphism is quite common, with worldwide frequencies of the two alleles being approximately equal.

Some of the inventors of the present invention demonstrated previously that individuals with diabetes mellitus (DM) who are homozygous for the Hp 2 allele (Hp 2-2) are at increased risk for myocardial infarction, stroke and cardiovascular death as compared with DM individuals homozygous for this polymorphism (Hp 1-1) (Blum et al., Pharmacogenomics., 2008, 9(8):989-91). In addition, it has been shown by some of the inventors of the present invention and their coworkers that the antioxidant, vitamin E, reduces the risk for cardiovascular diseases in Hp 2-2 genotype individuals with diabetes mellitus (Milman et al., Artherioscler. Thromb. Vasc. Biol., 2008, 28:341-347; Blum et al., Pharmacogenomics. 2010, 11(5):675-84; and Blum et al., Atherosclerosis, 2010, 211:25-27). The mechanism by which vitamin E exerts its selective therapeutic effect in Hp 2-2 genotype, but not in the Hp 2-1 genotype is disclosed by some of the inventors of the present invention (Farbstein et al., Atherosclerosis. 2011; 219(1): 240-244).

U.S. Pat. Nos. 6,251,608; 6,613,519 and 6,599,702, by one of the inventors of the present invention, disclose methods of evaluating a risk of a diabetic patient to develop a vascular complication based on the haptoglobin phenotype of the diabetic patient, wherein the risk is decreased in patients with haptoglobin 1-1 phenotype as compared to patients with haptoglobin 2-1 or haptoglobin 2-2 phenotypes.

U.S. Pat. No. 7,608,393 and U.S. Patent Publication Nos. 2008/0044399, 2004/0229244 and 2007/0218462 disclose the potential of a diabetic patient to benefit from an anti-oxidant therapy, such as, vitamin E, for treatment of a vascular complication, based on the haptoglobin phenotype of the diabetic patient, whereby a patient having a haptoglobin 2-2 phenotype benefits from the anti oxidant therapy more than a patient having a haptoglobin 2-1 phenotype or a patient having a haptoglobin 1-1 phenotype.

U.S. Patent Application Publication No. 2009/0137617 by one of the inventors of the present invention, discloses a method of determining the potential of a subject having a cardiovascular disorder to benefit from administration of vitamin E in combination with a statin.

In addition, U.S. Patent Publication Nos. 2009/0074740, 2009/0246770 and 2010/0041059 by one of the inventors of the present invention disclose the use of haptoglobin genotyping in diagnosis and treatment of defective reverse cholesterol transport (RCT), methods of reducing risk of developing cardiovascular complications in diabetic patients and methods of determining a potential of a diabetic patient to benefit from antioxidant therapy for treatment of a vascular complication, respectively.

Furthermore, U.S. Patent Publication No. US2011/0294145 discloses antibodies and methods of using same for detecting haptoglobin phenotype.

There remains an unmet medical need for providing beneficial approaches for preventing, attenuating or ameliorating manifestation of CVD.

### SUMMARY OF THE INVENTION

The invention is directed to compositions and methods of treating a cardiovascular disease, disorder or condition in a subject in need thereof, the methods comprise as a first step, determining the haptoglobin phenotype of a patient. According to one embodiment, the methods comprise, further steps of identifying a patient having haptoglobin 1-1 phenotype and treating the patient with a therapeutically active component capable of raising high density lipoprotein (HDL). According to another embodiment, the methods further comprises the steps of identifying a patient having haptoglobin 2-2 phenotype and treating the patient with a combination therapy comprising a first pharmaceutical composition comprising an antioxidant and a second pharmaceutical composition comprising a therapeutically active component capable of raising HDL.

Recent clinical data of treatments aimed at raising HDL levels in the blood circulation (e.g., treatment with CETP inhibitors such as Torcetrapib®), has implicated that not only do those agents not show improved efficacy in the condition of CVD patients, but also those agents are deleterious to considerable number of patients. In accordance with those findings certain clinical studies aimed at raising HDL levels were halted.

The inventors of the present invention have unexpectedly found that treatment with agents aimed at raising HDL may in fact be efficacious and that the beneficial effect or course of HDL therapy should be determined according to the haptoglobin (Hp) genotype or phenotype of the patient. As exemplified herein below, treatment of patients having a haptoglobin 1-1 phenotype with an agent capable of raising HDL (i.e., the niacin Niaspan®) decreased rates of complications (e.g., heart attack) or deaths associated with CVD. In contrast, in patients having either haptoglobin 2-1 or haptoglobin 2-2 phenotype treatment with Niaspan® increased rates of CVD events. According to some embodiments, the patient is afflicted with diabetes mellitus (DM).

Taken together, the present invention provides for the first time an exclusion criterion for treatment of patients with HDL raising therapy, the criterion being the patients' haptoglobin phenotype—patients having haptoglobin 1-1 phenotype may benefit from treatment with agents capable of raising HDL while other patients having 2-2 phenotype would have to be treated with a combination of an antioxidant and an agent capable of raising HDL, wherein these agents can be administered separately, sequentially or simultaneously. According to some embodiments, the patient is afflicted with diabetes mellitus (DM).

According to a first aspect, the present invention provides a method of treating a cardiovascular disease or disorder in a patient, comprising:
a. determining the haptoglobin phenotype of a patient;
b. identifying a patient having a haptoglobin 1-1 phenotype; and
c. administering to the patient having a haptoglobin 1-1 phenotype a pharmaceutical composition comprising a therapeutically active component capable of raising HDL, thereby treating the disease or disorder. According to some embodiments, the patient is afflicted with diabetes mellitus (DM). In some embodiment of the invention, the method further comprises the step of assessing HDL functionality in the subject in need wherein if the HDL of the subject is dysfunctional the subject is treated with a first pharmaceutical composition comprising an antioxidant and a second pharmaceutical composition comprising a therapeutically active component capable of raising HDL.

According to a first aspect, the present invention provides a method of treating a cardiovascular disease or disorder in a patient, comprising:
a. determining the haptoglobin phenotype of a patient;
b. identifying a patient having a haptoglobin 2-1 phenotype; and
c. administering to the patient having a haptoglobin 2-1 phenotype a pharmaceutical composition comprising a therapeutically active component capable of raising HDL, thereby treating the disease or disorder. According to some embodiments, the patient is afflicted with diabetes mellitus (DM). In some embodiment of the invention, the method further comprises the step of assessing HDL functionality in the subject in need wherein if the HDL of the subject is dysfunctional the subject is treated with a first pharmaceutical composition comprising an antioxidant and a second pharmaceutical composition comprising a therapeutically active component capable of raising HDL.

According to another embodiment, treating the cardiovascular disease comprises at least one of preventing deterioration of the cardiovascular disease and reducing the risk for pathology resulting from the cardiovascular disease.

According to yet another embodiment, treating comprises raising the level of HDL in the blood of the patient having a haptoglobin 1-1 or 2-1 phenotype to at least 50 mg/dL.

According to yet another embodiment, the cardiovascular disease is selected from the group consisting of: coronary atherosclerosis, dyslipidemia, type II dyslipidemia, hypercholesterolemia and myocardial infarction.

According to yet another embodiment, the therapeutically active component is a cholesterylester transfer protein (CETP) inhibitor.

According to yet another embodiment, the CETP inhibitor is selected from the group consisting of: S-[2-({[1-(2-ethylbutyl)cyclohexyl]carbonyl}amino)phenyl] 2-methylpropanethioate (Dalcetrapib®; JTT-705), ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate (Torcetrapib®), Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cyclohexanecarboxylic acid (Evacetrapib®; LY2484595), DRL-17822 and (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one (Anacetrapib®).

According to yet another embodiment, the therapeutically active component is a fibrate.

According to yet another embodiment, the therapeutically active component comprises niacin (pyridine-3-carboxylic acid).

According to yet another embodiment, the pharmaceutical composition is in a form selected from the group consisting of: long acting formulation, controlled release formulation, sustained release formulation, bioadhesive formulation, mucoadhesive formulation and slow release formulation.

According to another aspect, the present invention provides a method of treating a cardiovascular disease or disorder in a patient, comprising:
a. determining the haptoglobin phenotype of a patient;
b. selecting a patient having a haptoglobin 2-2 phenotype; and
c. administering to the patient having a haptoglobin 2-2 phenotype a first pharmaceutical composition comprising an antioxidant and a second pharmaceutical composition comprising a therapeutically active component capable of raising HDL, thereby treating the disease or disorder. According to some embodiments, the patient is afflicted with diabetes mellitus (DM). In some embodiments, the method further comprises the step of assessing HDL functionality in the subject in need wherein if the HDL of the subject is functional the subject is treated with a pharmaceutical composition comprising a therapeutically active component capable of raising HDL.

According to another embodiment, treating the cardiovascular disease comprises at least one of preventing deterioration of the cardiovascular disease and reducing the risk for the cardiovascular disease.

According to yet another embodiment, treating comprises raising the level of HDL in the blood of the patient having a haptoglobin 2-2 phenotype to at least 50 mg/dL.

According to yet another embodiment, the cardiovascular disease comprises at least one of coronary atherosclerosis, dyslipidemia, type II dyslipidemia, hypercholesterolemia and myocardial infarction.

According to yet another embodiment, the therapeutically active component capable of raising HDL is a CETP inhibitor.

According to yet another embodiment, the CETP inhibitor is selected from the group consisting of: S-[2-({[1-(2-ethylbutyl)cyclohexyl]carbonyl}amino)phenyl] 2-methylpropanethioate, ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate, Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cyclohexanecarboxylic acid and (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one.

According to yet another embodiment, the therapeutically active component capable of raising HDL is niacin.

According to yet another embodiment, the antioxidant is a tocopherol.

According to yet another embodiment, the tocopherol is selected from the group consisting of alpha-tocopherol, d-alpha-tocopherol, beta-tocopherol, gamma-tocopherol and tocotrienol.

According to yet another embodiment, the first pharmaceutical composition is administered prior to the second pharmaceutical composition.

According to yet another embodiment, the first pharmaceutical composition and the second pharmaceutical composition are administered simultaneously.

According to yet another aspect, the present invention comprises a pharmaceutical composition comprising a therapeutically active component capable of raising HDL for the treatment of a cardiovascular disease or disorder in a patient having a haptoglobin 1-1 phenotype. According to some embodiments, the patient is afflicted with diabetes mellitus (DM).

According to yet another aspect, the present invention provides a first pharmaceutical composition comprising an antioxidant and a second pharmaceutical composition comprising a therapeutically active component capable of raising HDL for the treatment of a cardiovascular disease or disorder in a patient having a haptoglobin 2-2 phenotype. According to some embodiments, the patient is afflicted with diabetes mellitus (DM).

According to yet another aspect, the present invention provides the use of a pharmaceutical composition comprising a therapeutically active component capable of raising HDL for the treatment of a cardiovascular disease or disorder in a patient having a haptoglobin 1-1 phenotype. According to some embodiments, the patient is afflicted with diabetes mellitus (DM).

According to yet another aspect, the present invention provides the use of a first pharmaceutical composition comprising an antioxidant, and a second pharmaceutical composition comprising a therapeutically active component capable of raising HDL for the treatment of a cardiovascular disease or disorder in a patient having a haptoglobin 2-2 phenotype. According to some embodiments, the patient is afflicted with diabetes mellitus (DM).

According to yet another aspect, the present invention provides a kit for treating a cardiovascular disease or disorder in a patient in need thereof, comprising means for determining the haptoglobin phenotype of a patient, a pharmaceutical composition comprising a therapeutically active component capable of raising HDL, and instructions for use for the treatment of a patient that had been identified as having a haptoglobin 1-1 phenotype. According to some embodiments, the patient is afflicted with diabetes mellitus (DM).

According to yet another aspect, the present invention provides a kit for treating a cardiovascular disease or disorder in a patient in need thereof, comprising means for determining the haptoglobin phenotype of a patient, a first pharmaceutical composition comprising an antioxidant, a second pharmaceutical composition comprising a therapeutically active component capable of raising HDL, and instructions for use for the treatment of a patients having a haptoglobin 2-2 phenotype. According to some embodiments, the patient is afflicted with diabetes mellitus (DM).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In some embodiments of the invention there is provided a method of determining the functionality of HDL in a subject comprising the steps of: treating a serum or a plasma sample obtained from a subject to obtain apo-B depleted serum sample; adding oxidation-sensitive agent; calculating total oxidation of the oxidation-sensitive agent; depleting HDL from the depleted apo-B serum or plasma sample by immunoprecipitation; calculating the difference between the oxidation of the oxidation-sensitive agent slope after HDL depletion and the total oxidation slope of the oxidation-sensitive agent before HDL depletion; wherein positive values for the difference indicate that HDL is functional in that sample as an antioxidant and negative values for the difference indicate that HDL is functional in that sample as a pro-oxidant.

In some embodiments of the invention, the treating of the serum or the plasma sample to obtain apo-B depleted serum sample is by treating a serum sample obtained from a subject with polyethylene glycol (PEG).

In some embodiments of the invention, wherein the oxidation-sensitive agent is dihydrorhodamine.

In some embodiments of the invention, the total oxidation is calculated by determining the rate of DHR oxidation (fluorescent units (FU)/min) after subtracting the rate of DHR oxidation observed using the same conditions but in the absence of serum.

In some embodiments of the invention, the immunoprecipitation is performed by using anti-human apoA1 antibody and Sepharose.

In some embodiments of the invention, the Sepharose is protein A/G Sepharose.

In some embodiments of the invention, there is provided a kit for determining the functionality of HDL in a subject comprising: oxidation-sensitive agent; means for depleting apo-B serum from a serum sample; means for immunoprecipitation of HDL and a leaflet explaining the steps of the method for determining the functionality of HDL in a subject. In some embodiments of the invention, the means for obtaining apo-B depleted serum sample is polyethylene glycol (PEG). In some embodiments of the invention, the oxidation-sensitive agent is dihydrorhodamine—(DHR). In some embodiments of the invention, the means for immunoprecipitation is anti-human apoA1 antibody and Sepharose.

In some embodiments of the invention, there is provided a pharmaceutical composition comprising a therapeutically active component capable of raising HDL for the treatment of a cardiovascular disease or disorder in a patient having a haptoglobin 1-1 or 2-1 phenotype or a patient having haptoglobin 2-2 phenotype which HDL was identified as functional.

In some embodiments of the invention, there is provided a first pharmaceutical composition comprising an antioxidant and a second pharmaceutical composition comprising a therapeutically active component capable of raising HDL for the treatment of a cardiovascular disease or disorder in a patient having a haptoglobin 2-2 phenotype or a patient having haptoglobin 1-1 or 2-1 phenotype which HDL was identified as dysfunctional.

In some embodiments of the invention, the invention provides use of pharmaceutical composition comprising a therapeutically active component capable of raising HDL for the treatment of a cardiovascular disease or disorder in a patient having a haptoglobin 1-1 or 2-1 phenotype or patient having haptoglobin 2-2 phenotype which HDL was identified as functional.

In some embodiments of the invention, the invention provides use of a first pharmaceutical composition comprising an antioxidant, and a second pharmaceutical composition comprising a therapeutically active component capable of raising HDL for the treatment of a cardiovascular disease or disorder in a patient having a haptoglobin 2-2 phenotype or a patient having haptoglobin 1-1 or 2-1 phenotype which HDL was identified as dysfunctional.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIG. 1: FIG. 1A is a bar graph showing that niacin increases RCT function in Hp 1-1 but not in Hp 2-2 individuals. RCT at study enrollment was significantly increased at study enrollment in Hp 1-1 compared to Hp 2-2 participants (p=0.001) and there was no significant difference in baseline RCT between participants randomized to niacin or placebo within a given Hp genotype. FIG. 1B is a graph showing change in RCT function stimulated by serum from Hp 1-1 and Hp 2-2 participants from baseline to one year on treatment in individuals randomized to placebo or niacin. RCT was significantly increased in Hp 1-1 individuals on niacin (p=0.019) but not in Hp 2-2 individuals on niacin (p=0.23). There was also no significant change in RCT in either placebo group. Forest plot shows mean and 95% CI for each study group.

FIG. 2: FIG. 2A is a bar graph showing total oxidation stimulated by ApoB depleted serum in each of the four study treatment groups at baseline. Total oxidation by ApoB depleted serum was significantly increased at study enrollment in Hp 2-2 compared to Hp 1-1 participants (p=0.002). FIG. 2B is a graph showing change in total oxidation in Hp 1-1 and Hp 2-2 participants from baseline to one year on treatment in individuals randomized to placebo or niacin. At one year total oxidation significantly decreased in the Hp 1-1 niacin group by 19.1±3.4 oxidation units while in the Hp 2-2 niacin group total oxidation was significantly increased by 13.4±5.0 oxidation units. There was no significant change in total oxidation in either the Hp 1-1 or Hp 2-2 placebo groups one year into the study. There was a highly significant interaction between the Hp genotype and niacin on total oxidation (p<0.0001). Forest plot shows mean and 95% CI for each study group.

FIG. 3 demonstrates DHR oxidation kinetics in the HDL antioxidant functional assay described in methods. Upper panel (FIG. 3A) shows a representative sample with preserved HDL antioxidant function: DHR oxidation (+HDL) slope in FU/min is 53 and without HDL is 66.4. HDL associated antioxidant is the difference in these slopes (FU/min) 66.4-53=+13.4 indicating in this sample the HDL is functioning as an antioxidant. Lower panel (FIG. 3B) shows a representative sample with dysfunctional HDL antioxidant function in which the HDL is acting as a prooxidant: DHR oxidation (+HDL) slope in FU/min is 144.6 and without HDL is 131.7. HDL associated antioxidant function (FU/min) is 131.7-144.6=−12.9 indicating in this sample the HDL is functioning as a prooxidant.

FIG. 4: FIG. 4A is a bar graph showing interaction between Hp genotype and niacin on HDL antioxidant function. HDL antioxidant function (expressed in units of DHR oxidation prevented by HDL) was measured in each of the four treatment groups at baseline as described in methods with positive index indicating HDL was acting as an antioxidant and a negative index indicating that the HDL was acting as a pro-oxidant. At baseline HDL antioxidant function was significantly better in Hp 1-1 vs Hp 2-2 participants (p<0.001). FIG. 4B is a graph showing the change in HDL antioxidant function in Hp 1-1 and Hp 2-2 participants from baseline to one year on treatment in individuals randomized to placebo or niacin. At one year HDL antioxidant function increased in the Hp 1-1 niacin group by 10.8±2.3 units while in the Hp 2-2 niacin group antioxidant function was significantly decreased by 8.3±2.9 units. There was no significant change in HDL antioxidant function in either the Hp 1-1 or Hp 2-2 placebo groups one year into the study. There was a highly significant interaction between the Hp genotype and niacin on HDL antioxidant function (p<0.0001). Forest plot shows mean and 95% CI for each study group.

### DETAILED DESCRIPTION OF THE INVENTION

The present invention is directed to methods of treating a cardiovascular disease/disorder in a patient in need thereof, said methods comprising steps of determining and identifying haptoglobin 1-1 phenotype of a patient and administering to the patient a therapeutically active component capable of raising HDL. The invention further relates to methods of treating cardiovascular disease/disorder in patients having haptoglobin 2-2 phenotype comprising administering to a patient determined and identified as having a haptoglobin 2-2 phenotype a combination therapy comprising a first pharmaceutical composition comprising an antioxidant and a second pharmaceutical composition comprising a therapeutically active component capable of raising HDL.

While reducing the present invention to practice, as is exemplified in the Examples section that follows, CVD patients having Hp 1-1 phenotype benefit from treatment with an agent capable of raising HDL levels in the blood (i.e., Niaspan®), whilst in the rest of the DM patients, namely in patients having either HP 2-1 or 2-2 phenotype such a therapy is deleterious.

Several recently published clinical trials, failed to indicate any benefit of agents capable of raising HDL (such as CETP inhibitors and niacins) for CVD therapy. In fact, a large body of evidence even indicated that treatment with CETP inhibitors is deleterious in some cases. Specifically, a study, designed to determine the clinical outcome of raising HDL by treatment with the CETP inhibitor Torcetrapib® in combination with Atorvastatin® was terminated abruptly and unexpectedly after a little more than a year of treatment, because of an excess of deaths in the Torcetrapib®/Atorvastatin® versus Atorvastatin® groups (82 versus 51, respectively). Increases in heart failure, angina, and revascularization procedures were also observed (Alan R. Tall et al., Arteriosclerosis, Thrombosis, and Vascular Biology; 2007; 27: 257-260). In further study, testing efficacy of the CETP inhibitor Dalcetrapid®, in patients who had a recent acute coronary syndrome, Dalcetrapib® had been found to increase HDL cholesterol levels but did not reduce the risk of recurrent cardiovascular events (Gregory G. Schwartz, et al., N. Engl. J. Med., 2012; 367:2089-2099). In yet a further trial involving patients with established, non acute cardiovascular disease Niaspan® plus Simvastatin® as compared with Simvastatin® alone was associated with significant increases in HDL cholesterol levels and decreases in triglyceride levels, but there was no significant reduction in the primary composite end point of cardiovascular events over a mean follow-up period of 36 months (The AIM-HIGH investigators, N. Engl. J. Med., 2011; 365(24): 2255-2267).

In contrast to those results which establish medications that raise HDL as inappropriate in treating CVD, the present inventors have, for the first time, demonstrated that those agents may indeed benefit with CVD patients, specifically, with CVD patients afflicted also with DM. The inventors of the present invention have now found and disclose that treatment with agents capable of raising HDL should be determined based on the Hp phenotype or genotype of the patient. That is to say, following Hp genotype or phenotype determination, patients' selection should be made and those patients with the Hp 2-2 phenotype should be first treated an antioxidant while patients having Hp 1-1 phenotype may be safely treated with an agent capable of raising HDL.

Thus, according to one aspect, the present invention provides a method for treating a cardiovascular disease or disorder in a patient, comprising the steps:
a. determining the haptoglobin phenotype of a patient;
b. identifying a patient having a haptoglobin 1-1 phenotype; and
c. administering to the patient having a haptoglobin 1-1 phenotype a pharmaceutical composition comprising a therapeutically active component capable of raising HDL, thereby treating the disease or disorder. According to some embodiments, the patient is afflicted with diabetes mellitus (DM).

Thus, according to one aspect, the present invention provides a method for treating a cardiovascular disease or disorder in a patient, comprising the steps:
d. determining the haptoglobin phenotype of a patient;
e. identifying a patient having a haptoglobin 2-1 phenotype; and
f. administering to the patient having a haptoglobin 2-1 phenotype a pharmaceutical composition comprising a therapeutically active component capable of raising HDL, thereby treating the disease or disorder. According to some embodiments, the patient is afflicted with diabetes mellitus (DM).

According to another aspect, the present invention provides a method of treating a cardiovascular disease or disorder in a patient, comprising:
a. determining the haptoglobin phenotype of a patient;
b. selecting a patient having a haptoglobin 2-2 phenotype; and
c. administering to the patient having a haptoglobin 2-2 phenotype a first pharmaceutical composition comprising an antioxidant and a second pharmaceutical composition comprising a therapeutically active component capable of raising HDL, thereby treating the disease or disorder. According to some embodiments, the patient is afflicted with diabetes mellitus (DM).

As used herein, the term “cardiovascular disease” is interchangeable with the term “heart disease” and refers to any disease, disorder or condition that involves the heart, the blood vessels (arteries, capillaries, and veins) or both. The term includes any disease that affects the cardiovascular system, principally cardiac disease, vascular diseases of the brain and kidney, and peripheral arterial disease.

According to some embodiments, the CVD is selected from, but is not limited to, the group consisting of: dyslipidemia, type II dyslipidemia, hypercholesterolemia, myocardial infarction (also known as, heart attack), coronary artery disease (also known as, coronary heart disease and ischaemic heart disease; e.g., atherosclerosis), cardiomyopathy, hypertensive heart disease, heart failure, cardiac dysrhythmias, inflammatory heart disease (e.g., endocarditis, inflammatory cardiomegaly, and myocarditis), valvular heart disease, cerebrovascular disease, peripheral arterial disease, congenital heart disease (CHD), and rheumatic heart disease. Each possibility represents a separate embodiment of the invention.

According to some embodiments, the patient afflicted with the cardiovascular disease is a diabetic patient.

The term “treating” as used herein, includes, but is not limited to, any one or more of the following: abrogating, ameliorating, inhibiting, attenuating, blocking, suppressing, reducing, delaying, halting, alleviating or preventing symptoms associated with CVD, and/or CVD related pathology or condition.

The methods of the invention comprise the step of determining a haptoglobin phenotype of a patient afflicted with a cardiovascular disease. In humans, a common polymorphism of the haptoglobin gene, characterized by alleles Hp 1 and Hp 2, gives rise to structurally and functionally distinct haptoglobin protein phenotypes, known as Hp 1-1, Hp 2-1, and Hp 2-2.

According to the embodiments of the invention, “determining a haptoglobin phenotype” is interchangeable with “determining a haptoglobin genotype” and includes either analysis of at least one of the gene, mRNA or protein of haptoglobin.

According to some embodiments, “determining the haptoglobin genotype” is accomplished by any suitable method known in the art including, but not limited to, a signal amplification method, a direct detection method and detection of at least one sequence change.

According to some embodiments, the signal amplification method amplifies a molecule selected from the group consisting of a DNA molecule and an RNA molecule. Suitable signal amplification methods include, but are not limited to, Polymerase Chain Reaction (PCR), Ligase Chain Reaction (LCR, also known as Ligase Amplification Reaction (LAR)), Self-Sustained Synthetic Reaction (3SR/NASBA) or a Q-Beta (Q.beta.) Replicase reaction. Each possibility represents a separate embodiment of the invention.

Suitable direct detection methods include, but are not limited to, a Cycling Probe Reaction (CPR) and a branched DNA analysis.

Suitable methods of detection of at least one sequence change include, but are not limited to, Restriction Fragment Length Polymorphism (RFLP) analysis, Allele Specific Oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

Determination of a haptoglobin phenotype may also be accomplished, by analyzing the protein products of the haptoglobin gene, or portions thereof. Such analysis is often accomplished using an immunological detection method which utilizes antibodies specific to at least one of the two haptoglobin alleles. Suitable immunological detection methods include, but are not limited to, a radio-immunoassay (RIA), an Enzyme Linked Immunosorbent Assay (ELISA), a western blot, an immunohistochemical analysis, and Fluorescence Activated Cell Sorting (FACS).

According to some embodiments, analyzing the protein products of the haptoglobin gene may be carried out with an antibody or an antigen-binding fragment thereof of an anti-haptoglobin antibody that binds with greater affinity to a one haptoglobin isoform than to the second haptoglobin isoform. The anti-haptoglobin antibody or antigen-binding fragment thereof may, according to one embodiment, bind with greater affinity to Hp 2-2 than to Hp 2-1, and with greater affinity to Hp 2-1 than to Hp 1-1. According to another embodiment, the anti-haptoglobin antibody or antigen-binding fragment thereof may bind with greater affinity to Hp 1-1 than to Hp 2-1, and with greater affinity to Hp 2-1 than to Hp 2-2. Such antibodies or antigen-binding fragment thereof may be monoclonal or polyclonal. Each possibility represents a separate embodiment of the invention.

According to some embodiments, the antibodies or antigen-binding fragment thereof, may be humanized or chimeric. According to some embodiments, the antibody may be a scFv antibody. According to some embodiments, any of the aforementioned anti-haptoglobin antibodies can further comprise at least one compound or polypeptide selected from a detectable label or a reporter. By way of non-limiting example, the detectable label is an enzyme such as horseradish peroxidase or alkaline phosphatase.

According to some embodiments, determining a haptoglobin phenotype of a subject comprises, contacting a biological sample of the subject with an anti-haptoglobin antibody or an antigen binding fragment thereof, forming a bound complex between the anti-haptoglobin antibody or fragment and haptoglobin in the biological sample; quantitatively determining a binding affinity between the haptoglobin and the anti-haptoglobin antibody or fragment; and comparing the quantitatively determined binding affinity with a value obtained from a quantitatively determined binding affinity of the anti-haptoglobin antibody or antigen binding fragment thereof to an isolated Hp 1-1, Hp 2-1, or Hp 2-2 isoform, wherein the binding affinity determined is indicative of the Hp isoform type, thereby determining the haptoglobin phenotype in the subject. According to some embodiments, “determining the haptoglobin phenotype or genotype”, may be effected using any suitable biological sample derived from the examined individual, including, but not limited to, a blood sample, a saliva sample, or other samples of body secretion, such as, urine and tears. Each possibility represents a separate embodiment of the invention.

According to one embodiment, the biological sample is plasma and the plasma is processed to produce serum.

Further in additional study, the AIM HIGH substudy, it was shown that the benefit or harm from HDL raising therapy with niacin on HDL metrics of RCT and antioxidant function may depend on the Hp genotype. In both Hp 1-1 and Hp 2-2 DM individuals niacin was associated with a significant increase in HDL mass, however only in Hp 1-1 participants was this translated into an improvement in HDL function. These data showing a pharmacogenomics interaction between the Hp genotype and niacin on HDL metrics may provide an explanation for the failure of HDL raising therapy, specifically niacin, to improve HDL functional metrics reduce CVD when given indiscriminately.

The HDL antioxidant assay described herein in Example 2 is a novel, robust and facile measurement of HDL function. The method described here is unique in allowing the assessment of this parameter for HDL alone (as opposed to ApoB depleted serum) in multiple patient samples simultaneously. The lack of an effect on this parameter in the placebo group provides high confidence as the reliability of the results with niacin. Remarkably, these data show that in over 40% of all Hp 2-2 participants, the HDL is paradoxically acting as a prooxidant. It is suggested that raising HDL in these Hp 2-2 participants with prooxidative HDL at baseline would be harmful.

The interaction between the Hp genotype and niacin on HDL function is most likely related to differences in the trafficking of extracellular Hb by Hp 1-1 and Hp 2-2. Extracorpuscular Hb is rapidly bound to Hp and this Hp-Hb complex is cleared by the monocyte/macrophage CD163 receptor. It was shown that the Hp 2-2-Hb complex is cleared more slowly than the Hp 1-1-Hb particularly in the setting of DM (20 vs 100 min T_{1/2}). The Hp-Hb complex that is not cleared will bind to ApoA1 via a specific interaction with helix 6. It was previously proposed but no quantitative proof was provided that the HDL from Hp 2-2 DM individuals contains more Hb. The example shows that using a novel ELISA there is a greater than 4 fold increase in the amount of Hb associated with the HDL of Hp 2-2 DM individuals and that there is a significant interaction between the amount of Hb associated with HDL, and the Hp genotype on RCT HDL function. Hb associated with HDL has been shown to result in increasing scavenging of nitric oxide and thereby impair the vascular protective effects of HDL. The inventors have recently present evidence for decreased bioavailability of NO in Hp 2-2 DM individuals which may reflect the increased Hb associated with Hp 2-2 DM HDL. An additional factor at play is the ability of the Hp 1-1 and Hp 2-2 proteins to neutralize the oxidative effects of Hb and it was shown that the Hp 2-2 is a poor antioxidant, particularly against glycosylated Hb. Accordingly, Hp 2-2-Hb bound to HDL would be expected to display more pro-oxidative activity than an equivalent mass of Hb bound to Hp 1-1. Consistent with this increased pro-oxidative activity of Hp 2-2 HDL it was shown that the HDL of Hp 2-2 DM individuals contains significantly less vitamin E than that Hp 1-1 DM in the HAPE study (unpublished data).

While these data suggest that HDL raising therapy with niacin might provide benefit to Hp 1-1 individuals they also suggest that repairing or restoring normal function to Hp 2-2 HDL may allow niacin to provide clinical benefit. The inventors have recently demonstrated in three independent studies that vitamin E can restore and significantly improve RCT function in Hp 2-2 DM individuals.

In some embodiments of the invention, the invention also covers the use of assay of HDL functionality which may identify a subpopulation of Hp 1-1 who have dysfunctional HDL and would benefit from a combined treatments of antioxidant and an agent capable of raising HDL as well as identifying a subpopulation of Hp 2-2 whose HDL is functional and could benefit from HDL therapy immediately

In some embodiments, the assay of HDL functionality may also be used in any subject. In some embodiments, the assay may be also used if it is a men with HDL cholesterol levels that are more than 40 mg/dL (1.0 mmol/L) or if it is a women HDL with cholesterol levels are high than 50 mg/dL (1.3 mmol/L) or more in order to determine whether their HDL is functional or not.

Dysfunctional HDL is defined as HDL, which induces oxidation rather than inhibits oxidation as defined in example in the HDL antioxidant assay described below. People that are identified with a dysfunctional HDL may benefit from a combined treatment comprising the two drugs: an antioxidant and an agent capable of raising HDL levels.

The assay is measures the ability of an individuals HDL to promote or inhibit oxidation.

An individual is defined as having HDL which is dysfunctional if his/her HDL promotes rather than inhibits oxidation in HDL antioxidant assay.

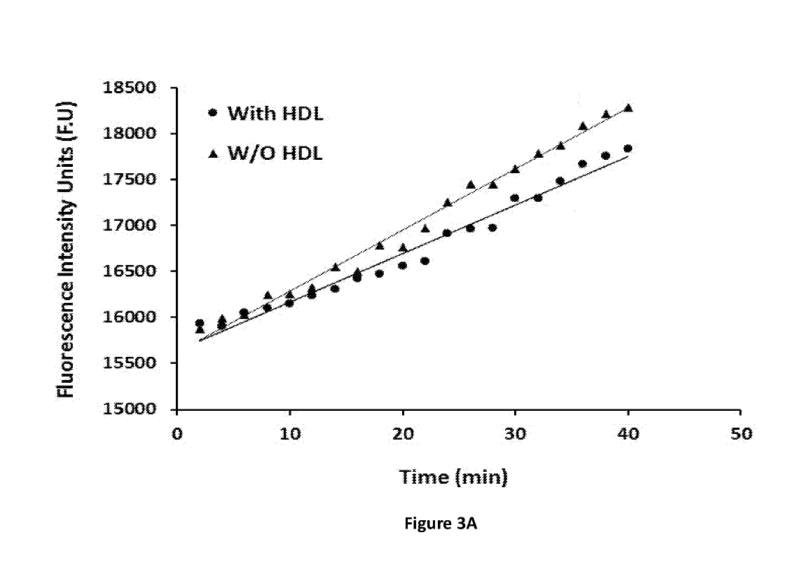

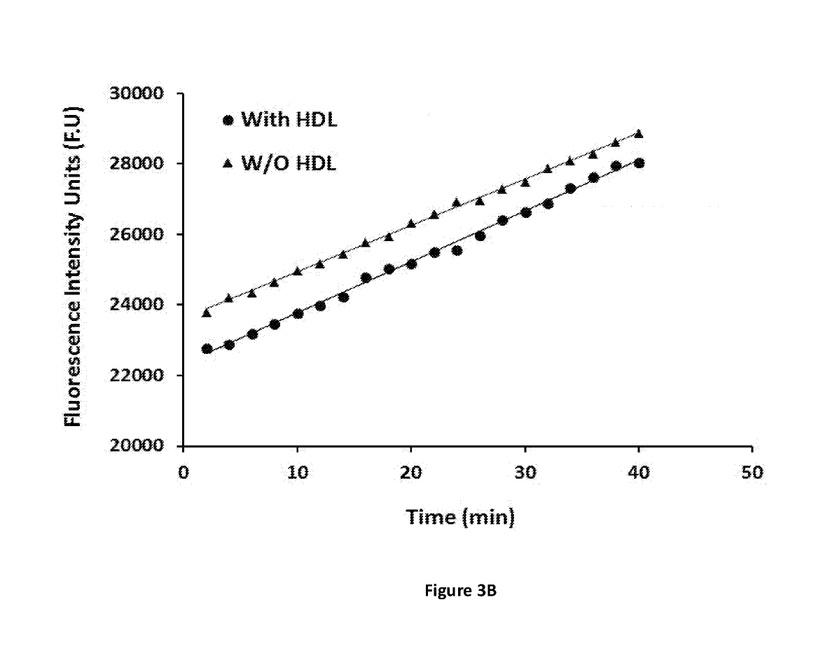

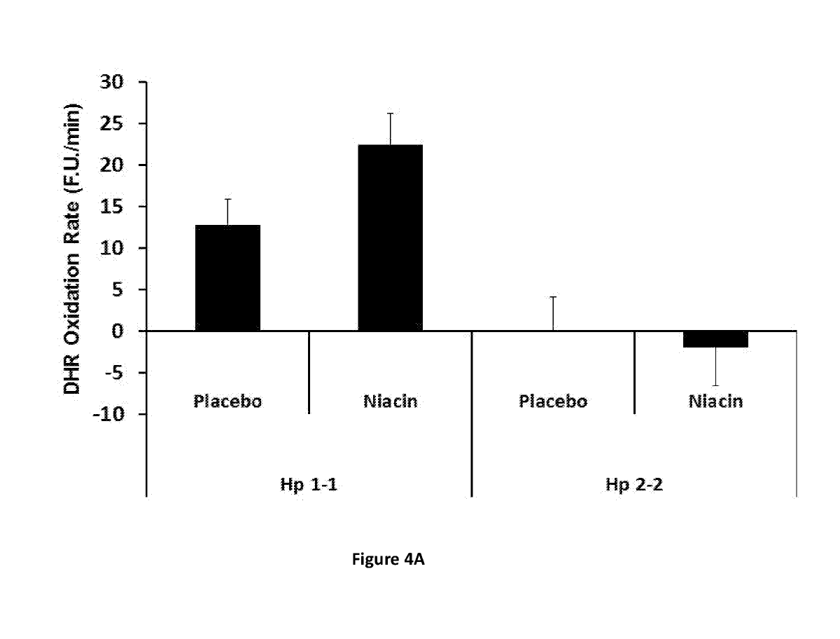

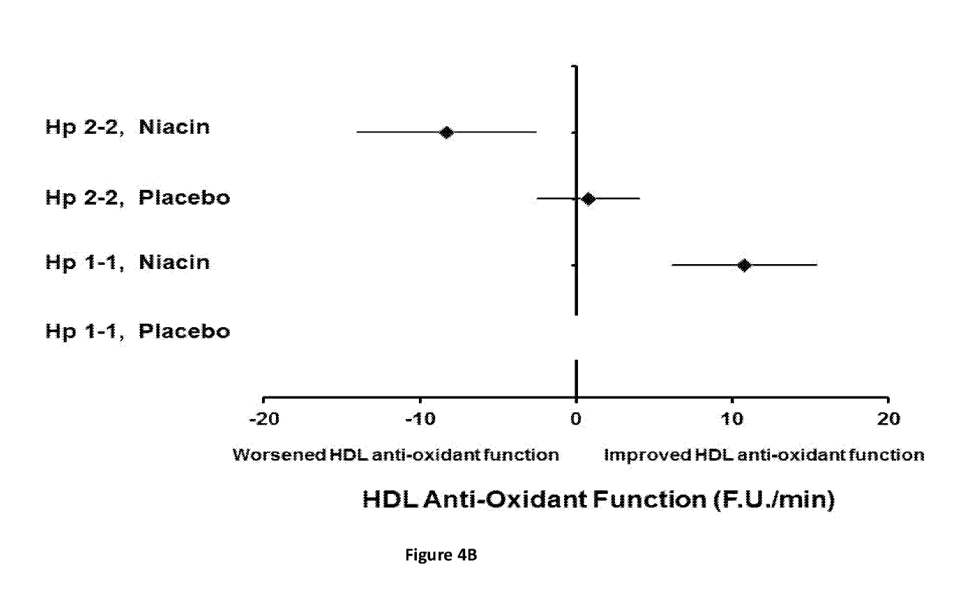

FIG. 3 provides a representative example of a patient with dysfunctional and functional HDL and how this is quantified in the assay. FIG. 4 provides the results from patients on niacin or placebo with either the Hp 1-1 or Hp 2-2 genotypes. The data show that HDL becomes more dysfunctional in Hp 2-2 individuals with niacin and more functional in Hp 1-1 with niacin. The assay provides a way of stratifiying individuals (regardless of Hp type) for a new metric of HDL function the ability to promote or inhibit oxidation.

In some embodiments, the HDL antioxidant assay is used for monitoring the efficiency of treatment is patients receiving medicines such as niacin, CETP inhibitors, HDL raising drugs, antioxidants, iron chelators/nitrites).

In some embodiments, the assay may be used for predicating the risk of developing CVD in a subject.

In some embodiments, the assay may be used as described herein to determine the treatment for an individual to in order to prevent or reduce the risk of CVD. In some embodiments, there is provided a method of determining the functionality of HDL in a subject comprising the steps of: treating a serum sample obtained from a subject to obtain apo-B depleted serum or plasma sample; adding oxidation-sensitive agent; calculating total oxidation of the oxidation-sensitive agent; depleting HDL from the depleted apo-B serum sample by immunoprecipitation; calculating the difference between the oxidation of the oxidation-sensitive agent slope after HDL depletion and the total oxidation slope of the oxidation-sensitive agent before HDL depletion; wherein positive values for the difference indicate that HDL is functional in that sample as an antioxidant and negative values for the difference indicate that HDL is functional in that sample as a pro-oxidant (serving to promote oxidation). In some embodiments, the step of treating of the serum sample to obtain apo-B depleted serum sample is by treating a serum sample obtained from a subject with polyethylene glycol (PEG). In some embodiments of the invention, the oxidation-sensitive agent fluorescent activated redox sensitive probe. In some embodiments, the fluorescent activated redox sensitive probe is dihydrorhodamine—(DHR). In some embodiments, it may be any agent which changes its biophysical properties in some way when it is oxidized in a way that can be monitored. —In some embodiments, the fluorescent activated redox sensitive probe become fluorescent when oxidized like DHR and there are other ways to detect oxidation of a substrate (such as a change in the absorption in a certain wavelength when the molecule is oxidized). In some embodiments of the invention, total oxidation is calculated by determining the rate of oxidation (fluorescent units (FU)/min) of the oxidation-sensitive agent after subtracting the rate of oxidation of the oxidation-sensitive agent observed using the same conditions but in the absence of serum. The measurement may be done in example by monitoring the oxidation using a fluorimeter. The immunoprecipitation may be performed in some embodiments by using anti-human apoA1 antibody and Sepharose, wherein the Sepharose according to some embodiments is protein A/G Sepharose.

In some embodiments, there is provided a kit for determining the functionality of HDL in a subject comprising: oxidation-sensitive agent; means for depleting apo-B serum from a serum sample; means for immunoprecipitation of HDL and a leaflet explaining the steps of the method for determining the functionality of HDL in a subject as described above.

In some embodiments of the invention, the means for obtaining apo-B depleted serum sample is polyethylene glycol (PEG).

In some embodiments of the invention, the means for immunoprecipitation is anti-human apoA1 antibody and Sepharose.

In some embodiments of the invention, the Sepharose is protein A/G Sepharose.

As used herein the term “therapeutically active component capable of raising HDL” refers to any agent, medicament, drug, administration of which results in an elevation of high-density lipoprotein (HDL) levels in the blood. High levels of HDL cholesterol are associated with a reduced risk of CVD, especially, coronary artery disease (CAD). HDL particles are known to “scour” the walls of blood vessels, cleaning out excess cholesterol that otherwise might have been deposited on blood vessels walls making plaques that cause CAD. The HDL cholesterol is then carried to the liver, where it is processed into bile, and secreted into the intestines and out of the body.

Typically, both HDL and LDL levels are measured in milligrams of cholesterol per deciliter (mg/dL) of blood or millimoles per liter (mmol/L). HDL cholesterol levels are thought to be impacted by genetics with women generally have higher HDL cholesterol levels than men. Men in which HDL cholesterol levels are less than 40 mg/dL (1.0 mmol/L) or women in which HDL cholesterol levels are less than 50 mg/dL (1.3 mmol/L) are considered as having low HDL levels and are thus classified as being at risk of having CVD. In contrast, HDL cholesterol levels of 60 mg/dL (1.6 mmol/L) or above is the desirable level.

According to some embodiments, the methods of treating CVD comprise raising the HDL levels in the blood of a patient to be at least 45 mg/dL, at least 50 mg/dL, at least 55 mg/dL or at least 60 mg/dL, irrespective of the gender of the patient. Each possibility represents a separate embodiment of the invention. According to one embodiment, treating CVD comprises raising the HDL levels in the blood of the patient to be at least 50 mg/dL. According to an alternative embodiment, the methods of treating CVD comprise raising the HDL levels in the blood of a man patient to be at least 45 mg/dL, at least 50 mg/dL, at least 55 mg/dL or at least 60 mg/dL. Each possibility represents a separate embodiment of the invention. According to a further embodiment, the methods of treating CVD comprise raising the HDL levels in the blood of a woman patient to be at least 55 mg/dL, or at least 60 mg/dL. Each possibility represents a separate embodiment of the invention.

As HDL cholesterol levels are thought to be influenced not only by the gender, but also by genetics and accordingly may vary in each individual, some researchers have proposed calculating the HDL levels by determining the ratio of total cholesterol (namely, LDL plus HDL) to HDL. In accordance with this embodiment, the calculation is as follows: the total cholesterol number is divided by the HDL number. The present invention encompasses the HDL calculations mentioned herein above and any other type of measurement or calculation of the HDL levels of a patient.

According to some embodiments, the term “raising” is interchangeable with increasing, or elevating. According to some embodiments, the methods of treating CVD comprise raising the HDL levels in the blood of a patient by at least 1.2, 1.4, 1.6, 1.8, or 2. Each possibility represents a separate embodiment of the invention.

According to some embodiments, the methods of treating CVD comprise raising the HDL levels in the blood of a patient by at least 10%, 20%, 30%, 40%, 50%, 60%, 70%, 80%, 90%, or 100%. Each possibility represents a separate embodiment of the invention.

According to some embodiments, the therapeutically active component capable of raising HDL is a CETP inhibitor.

As used herein the term “CETP inhibitor” refers to an agent, drug, or medicament, capable of inhibiting cholesterylester transfer protein (CETP). The term encompasses also derivatives or analogues of the CETP inhibitor. CETP normally transfers cholesterol from HDL cholesterol to very low density or low density lipoproteins (VLDL or LDL). Inhibition of this process results in higher HDL levels and reduces LDL levels.

According to some embodiments, the CETP inhibitor is selected from the group consisting of: S-[2-({[1-(2-ethylbutyl)cyclohexyl]carbonyl}amino)phenyl] 2-methylpropanethioate (Dalcetrapib®; JTT-705), ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate (Torcetrapib®), Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cyclohexanecarboxylic acid (Evacetrapib®; LY2484595), DRL-17822 and (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one (Anacetrapib®). Each possibility represents a separate embodiment of the invention.

According to some embodiments, the therapeutically active component capable of raising HDL is a fibrate.

As used herein the term “fibrate” denotes a class of amphipathic carboxylic acids. Fibrates are lipid-modifying agents, used to ameliorate a range of metabolic disorders, mainly hypercholesterolemia (high blood cholesterol levels). Treatment with fibrates usually results in a substantial decrease in plasma triglycerides and is usually associated with a moderate decrease in LDL cholesterol and an increase in HDL cholesterol concentrations. Exemplary fibrate formulations suitable in the context of the preset invention, include, but are not limited to, Bezafibrate (e.g. Bezalip®), Ciprofibrate (e.g. Modalim®), Clofibrate (e.g. Gallstones®), Gemfibrozil (e.g. Lopid@) and Fenofibrate (e.g. TriCor®). Each possibility represents a separate embodiment of the invention.

According to some embodiments, the therapeutically active component capable of raising HDL as is known to date is APOA1-mimetic peptide, LXR agonist (including T0901317 and GW3965), FXR agonist, Endothelial lipase inhibitor (including derivatives of sulphonylurea and boronic acid), MicroRNA antagonist (antagonists of miR-33), antisense nucleotide (ASOs targeting CETP and ApoCIII), APOA1 transcriptional upregulator, HDL mimetic prepared from recombinant APOA1, HDL mimetic manufactured from purified, authentic, human plasma APOA1 reconstituted with phospholipid, a naturally occurring mutated variant of the APOA1 protein, an oral APOA1 mimetic peptide (D-4F), recombinant human LCAT (the enzyme component of the RCT system), anti-CETP vaccine or CETP inhibitor (Cholesterylester transfer protein, such as Torcetrapib, Anacetrapib, Dalcetrapib, and Evacetrapib).

According to some embodiments, the naturally occurring mutated variant of the APOA1 protein is associated with a low frequency of cardiovascular disease. According to some embodiments, the HDL mimetic prepared from recombinant APOA1 is produced in mammalian cell expression systems complexed with phospholipids. According to some embodiments, the APOA1 transcriptional upregulator is an oral APOA1 transcriptional upregulator.

According to some embodiments, the therapeutically active component capable of raising HDL comprises niacin, a derivative or an analogue thereof.

As used herein, the term “niacin” denotes a B3 vitamin present in many food products such as, dairy products, eggs, enriched breads and cereals, fish, legumes, nuts and poultry. Niacin has been used since the 1950s in treatments attempting to lower elevated LDL cholesterol and triglyceride (fat) levels in the blood. The term is interchangeable with any alternative name or synonym known in the art including, but not limited to nicotinic acid, vitamin PP, pellagra-preventive, and anti-dermatitis factor. Also encompassed within the embodiments of the invention are formulations which comprise niacin and an additional active agent. Exemplary niacin formulations suitable in the context of the preset invention, include, but are not limited to Niaspan® (film-coated extended-release) and Tredaptive® (also known as Cordaptive®, a combination of niacin and laropiprant, a prostaglanding receptor antagonist).

As used herein the term “an analogue or a derivative thereof” includes suitable active variants of the CETP inhibitors described herein, such as, an analog or a modified CETP molecule. Chemical modification, in the context of the present invention includes modification with a chemical entity, group or moiety. Moreover, each particular compound, such as those described herein, may give rise to an entire family of analogues or derivatives having similar activity and, therefore, usefulness according to the present invention. Likewise, a single compound, such as those described herein, may represent a single family member of a greater class of compounds useful according to the present invention. Accordingly, the present invention fully encompasses not only the compounds described herein, but analogues and derivatives of such compounds, particularly those identifiable by methods commonly known in the art and recognizable to the skilled artisan.

According to some embodiments, the present invention provides methods of treating a patient afflicted with a CVD and having a haptoglobin 2-2 phenotype, comprising administering a first and a second pharmaceutical composition, wherein the first pharmaceutical is an antioxidant.

As of the date of filing this application, clinical trials and meta analysis of antioxidants have provided mixed results as regards the beneficial effect of those agents in treating CVD. The inventors of the present invention, disclose and enable a novel therapeutic strategy to treat CVD. This strategy discloses that patients with haptoglobin 2-2 may indeed benefit from a combination therapy comprising the two drugs: an antioxidant and an agent capable of raising HDL levels.

As used herein, the term “an antioxidant” refers to natural substances that exist as vitamins, minerals and other compounds in foods. Theoretically, antioxidants are believed to prevent CVD by ameliorating free radicals that, without adequate amounts of antioxidants, oxidate LDL, thus contributing to creation of plaques in the blood vessels. The term includes, but is not limited to, vitamin E, vitamin C, alpha carotene, and beta carotene. Each possibility represents a separate embodiment of the invention.

According to one embodiment, the antioxidant is vitamin E. The term “vitamin E” refers to a group of fat-soluble compounds including the many isomers and derivatives of tocopherols, tocopheryls and tocotrienols, which have vitamin E activity. Each possibility represents a separate embodiment of the invention. According to one embodiment, the vitamin E is tocopherol. “Tocopherols” are a class of chemical compounds of which many have vitamin E activity. It is a series of organic compounds consisting of various methylated phenols.

According to one embodiment, the tocopherol is selected from the group consisting of alpha-tocopherol, d-alpha-tocopherol, beta-tocopherol, gamma-tocopherol and tocotrienol. Each possibility represents a separate embodiment of the invention.

The present invention relates to methods of treating CVD comprising administering either a pharmaceutical composition comprising a therapeutically active component capable of raising HDL to a subject having a haptoglobin 1-1 phenotype or a first pharmaceutical composition comprising an antioxidant and a second pharmaceutical composition comprising a therapeutically active component capable of raising HDL to a patient having a haptoglobin 2-2 phenotype.

As used herein the term “pharmaceutical composition” refers to a preparation of one or more of the active component capable of raising HDL or an antioxidant, with other components such as pharmaceutically acceptable carriers, excipients or diluents. The purpose of a pharmaceutical composition is to facilitate administration of a compound to a subject.

As used herein, the term “excipient” refers to an inert substance added to a pharmaceutical composition to further facilitate administration of the active ingredients. Examples, without limitation, of excipients include calcium carbonate, calcium phosphate, various sugars and types of starch, cellulose derivatives, gelatin, vegetable oils, and polyethylene glycols.

As used herein, the term “carrier” refers to any substance suitable as a vehicle for delivering of the active ingredients of the present invention (i.e., an active component capable of raising HDL or an antioxidant) to a suitable biological site or tissue. As such, carriers can act as a pharmaceutically acceptable excipient of the pharmaceutical composition of the present invention. Carriers may include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers).

According to some embodiments, the pharmaceutical composition is in a form selected from the group consisting of: long acting formulation, controlled release formulation, sustained release formulation, bioadhesive formulation, mucoadhesive formulation and slow release formulation.

According to some embodiments, the composition comprising the active ingredients of the invention may further comprise an additional active ingredient or agent.

The term “patient” is interchangeable with the term “subject” and includes humans, and animals. According to one embodiment, the subject is a human subject. According to another embodiment, the subject is a diabetic subject.

The term “subject” as used herein, includes, for example, a subject who has been diagnosed to be afflicted with a CVD or subjects that have been refractory to the previous treatments. Also encompassed within the present invention is a healthy subject having a risk of being affected with a CVD or a condition associated with CVD.

The pharmaceutical compositions of the invention may be administered by any suitable route and treatment regimen which result with the desired therapeutic effect. The preferred route of administration is systemic. A suitable systemic route of administration includes, but is not limited to, oral administration.

According to some embodiments, the first and second pharmaceutical compositions of the invention may be administered separately, wherein a time interval is taken between administrations. In accordance with those embodiments, the first pharmaceutical composition, namely an antioxidant is administered prior to the second pharmaceutical composition, namely, the therapeutically active component capable of raising HDL.

According to some embodiments, the first pharmaceutical composition is administered prior to the second pharmaceutical and the time interval between treatments with said first and second pharmaceutical compositions may vary, being a time interval of a few days and up to a few months. According to one embodiment, the time interval is two months.

It is to be noted that the first and second pharmaceutical compositions may also be administered simultaneously.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1: The Effect of Niacin on CVD—a Clinical Study

AIM-HIGH (clinicaltrials.gov NCT00120289) was a randomized double blind clinical trial which set out to test the HDL hypothesis—that raising HDL would reduce CVD events. This hypothesis was based on epidemiological data showing that individuals with higher HDL had lower CVD. AIM-HIGH set out to raise HDL via the drug niacin. The 3414 participants in AIM HIGH were randomly assigned to receive extended release niacin 1500-2000 g/day vs placebo. All participants received simvastatin. The primary endpoint was the first event of the composite of death from coronary heart disease, nonfatal myocardial infarction, ischemic stroke, hospitalization for an acute coronary syndrome, or symptom driven coronary or cerebral revascularization.

The trial was stopped after a mean follow up of 3 years due to a lack of efficacy. Niacin had successfully increase HDL from mean of 35 mg/dl to 42 mg/dl. The primary endpoint occurred in 282 patients in the niacin group (16.4%) vs 274 patients in the placebo group (16.2%); HR 1.02 95% 0.87-1.21.

Without being bound by any theory or mechanism, the inventors of the present invention assumed that one reason that niacin (and other HDL raising therapies with CETP inhibitors for example) may have failed to show benefit despite its ability to raise HDL is that raising HDL in individuals in whom HDL is dysfunctional (as defined by a diminished ability to stimulate reverse cholesterol transport and by the ability to promote oxidation of LDL and lipids) will increase CVD. HDL raising therapy is only appropriate in individuals in whom HDL function is preserved but HDL mass is decreased. As detailed above, the inventors have previously established that the Hp 2-2 genotype identifies DM individuals with dysfunctional proatherogenic HDL. In order to find out whether haptoglobin phenotype should be determined prior to treatment with niacin, serum samples from all DM participants of AIM HIGH (n=1140) from the coordinating center of AIM HIGH were obtained. Hp typing was performed by the inventors using polyacrylamide gel electrophoresis. Data on Hp typing was returned to the AIM HIGH coordinating center which provided the event rates in these participants segregated by Hp type and treatment (monotherapy with statins only or combination therapy with statins plus niacin). There were no differences in any demographic factor between treatment groups segregated by Hp status.

The results indicate that in patients having the 2-1 or 2-2 haptoglobin phenotype, the therapeutic effect of simvastatin together with niacin was 30% higher (OR=1.3) as compared to the therapeutic effect of lovastatin alone in the same sub population.

The results obtained from the clinical study are summarized in Table 1 hereinbelow.

TABLE 1

| | | Monotherapy | | Combination therapy | |
|---|---|---|---|---|---|
| | | (Simvastatin) | | (Simvastatin + Niacin) | |
| | | (N = 562) | | (N = 578) | |
| HP2-2 | N (%) | 203 | (36.1%) | 194 | (33.6%) |
| | N (%) Primary Event, | 33 | (16.3%) | 39 | (20.1%) |
| | % of N in this category | | | | |
| | Event Rate (95% CI) | 0.0532 | (0.0378, 0.0748) | 0.0676 | (0.0494, 0.0926) |
| | Hazard Ratio (95% CI) | | | 1.2626 | (0.7942, 2.0073) |
| | OR | | | 1.296 | (0.784, 1.808) |
| HP2-1 | N (%) | 257 | (45.7%) | 295 | (51.0%) |
| | N (%) Primary Event, | 46 | (17.9%) | 64 | (21.7%) |
| | % of N in this category | | | | |
| | Event Rate (95% CI) | 0.0582 | (0.0436, 0.0776) | 0.0714 | (0.0559, 0.0912) |
| | Hazard Ratio (95% CI) | | | 1.2278 | (0.8405, 1.7935) |
| | OR | | | 1.271 | (0.849, 1.693) |
| HP1-1 | N (%) | 96 | (17.1%) | 86 | (14.9%) |
| | N (%) Primary Event, | 23 | (24.0%) | 18 | (20.9%) |
| | % of N in this category | | | | |
| | Event Rate (95% CI) | 0.0803 | (0.0534, 0.1209) | 0.0769 | (0.0485, 0.1221) |
| | Hazard Ratio (95% CI) | | | 0.9600 | (0.5168, 1.7830) |
| | OR | | | 0.840 | (0.140, 1.540) |
| | OR for all DM patients | | | 1.186 | (0.892, 1.480) |
| | OR for all non-DM patients | | | 0.925 | (0.691, 1.160) |

### Example 2

A study sought to determine if there was a pharmacogenomic interaction between the Haptoglobin (Hp) genotype and niacin on changes in HDL functional metrics

### Background

HDL raising therapy has not shown any benefit on cardiovascular outcomes. Raising HDL in individuals in whom the HDL is dysfunctional may promote atherogenesis. Hp is a serum protein which is part of the HDL proteome. The Hp 1 and Hp 2 alleles at the Hp locus produce marked differences in Hp structure and function. HDL structure and function are abnormal in individuals with the Hp 2-2 genotype.

### Methods

Serum induced reverse cholesterol transport function and HDL antioxidant function were measured in 70 Hp 1-1 and 70 Hp 2-2 participants from the AIM HIGH study at baseline and at one year after randomization to placebo or niacin.

### Abbreviations and Acronyms

Hp = haptoglobin
Hb = hemoglobin
RCT = reverse cholesterol transport
DM = Diabetes Mellitus
OR = odds ratio
CI = confidence interval

### Methods

This AIM HIGH substudy was approved by the AIM HIGH steering committee and was designed to investigate whether the Hp phenotype influenced HDL functional metrics in DM participants in AIM HIGH. All 1140 DM participants in AIM HIGH were subjected to Hp phenotyping. Functional HDL metrics (RCT and antioxidant function) was evaluated at baseline and at one year of treatment in 70 Hp 1-1 and 70 Hp 2-2 DM individuals with half in the niacin and half in the placebo group. In 20 Hp 1-1 and 20 Hp 2-2 DM individuals the amount of Hb associated with the HDL at baseline was measured. Individuals performing all functional assays were blinded to treatment assignment of all samples used for this study.

### Hp Phenotyping

Hp phenotyping from serum was performed by polyacrylamide gel electrophoresis of Hb enriched serum which provides a fingerprint banding pattern for each Hp type.

### Measurement of HDL Cholesterol Efflux Capacity

Measurement of HDL efflux capacity was performed as previously described. Briefly, Murine macrophage J774 cells (1×10⁶/mL) were plated in 24-well plates for 48 hours, then washed and radiolabeled in DMEM containing 2 μCi/mL ³H-cholesterol. After an overnight incubation, cells were washed twice then incubated with 1 mL of DMEM containing 20 μL of serum in duplicates for 4 hours at 37° C. to permit efflux of ³H-cholesterol from the cells into the medium. Liquid scintillation counting was then determined in 500 μl of the medium collected from each well as well as in the cells after washing the cells twice with PBS and lysing them in 0.1 N NaOH. Cholesterol efflux capacity was determined as the percentage efflux of total counts per minute in the medium divided by the total counts per minute in the medium and in the cells and after subtraction of the nonspecific efflux obtained in cells incubated in the absence of serum.

### Measurement of Total Oxidation and HDL Anti-Oxidation Function

The amount of total oxidation and HDL anti-oxidant function were assessed in apolipoprotein (apo)-B depleted serum using the fluorescent and oxidation-sensitive agent dihydrorhodamine 123 (DHR). Apo-B depleted serum was prepared using polyethylene glycol (PEG) as previously described in Asztalos BF, de la Llera-Moya M, Dallal G E, Horvath K V, Schaefer E J, Rothblat G H. Differential effects of HDL subpopulations on cellular ABCA1- and SR-BI-mediated cholesterol efflux. J Lipid Res. 2005; 46:2246-2253

Briefly-serum samples were treated with PEG solution to precipitate apoB-containing lipoproteins by adding 40 parts PEG solution (20% PEG in 200 mM glycine buffer, pH 7.4) to 100 parts serum. After a 20 min incubation, the precipitate was removed by high-speed centrifugation (10,000 rpm, 30 min, 4° C.) to obtain the PEG supernatant containing the HDL lipoprotein fraction.

Duplicates of 20 μl of apo-B-depleted serum were transferred to clear-bottom 96-well plates, and 180 μl of iron-free Hepes-buffered saline (HBS) containing 50 μM DHR was then added. Immediately after the addition of DHR, the kinetics of fluorescence increase was followed in a BMG Galaxy Fluostar microplate reader at 37° C. with a 485/538 nm excitation/emission filter pair with repeated readings every 2 minutes up to 40 minutes. Total oxidation was calculated as the rate of DHR oxidation (fluorescent units (FU)/min) for each duplicate after subtracting the rate of DHR oxidation observed using the same conditions but in the absence of serum.

HDL anti-oxidation function represents a fraction of the total oxidation prevented by HDL (in which case HDL is behaving as an antioxidant) or provoked by HDL (in which case HDL is behaving as a pro-oxidant) within each sample. To calculate HDL anti-oxidant function, HDL was depleted by immunoprecipitation from apo-B-depleted serum using anti-human apoA1 antibody and protein A/G Sepharose, before duplicates of corrected volumes of remnant apo-B- and Apo-A-depleted serum were transferred to 96-well plates and the rate of DHR oxidation measured in the identical manner and in parallel with the measurement of total oxidation as described above. HDL anti-oxidant function for each sample was determined by calculating the difference between the DHR oxidation slope after HDL depletion and the total DHR oxidation slope before HDL depletion, with positive values for this difference indicating that HDL was functioning in that sample as an antioxidant and negative values for this difference indicating that HDL was functioning in that sample as a pro-oxidant (serving to promote oxidation).

### Quantitation of Hb Bound to HDL by ELISA

Isolation of HDL from Plasma by Immunoaffinity Chromatography for Use in the ELISA

HDL was obtained from 200 μl of plasma for these studies. 100 μl of anti-apoA1 sepharose in PBS with 0.5M NaCl was added to plasma in final volume of 1 cc. The plasma and anti-ApoA1 sepharose were mixed on rotary device for one hour at room temperature. The sepharose beads and solution were then transferred to a poly prep chromatography column (0.8×4 cm) (BioRad) and the solution was allowed to flow through. The beads were then washed with 10 ml of PBS with 0.5M NaCl and then 10 ml of PBS. 100 μl of 0.1 M Glycine pH 2.5 was added to the column and the eluate discarded (column void volume) and then the HDL was eluted with 3×100 ul of 0.1 M Glycine pH 2.5 into tubes containing 30 μl 1M Tris pH 9 and 30 μl fetal calf serum.

### Hb Sandwich-ELISA

96 well plate (Nunc immunoplate cat no 442404 from ThermoScientific) was coated with polyclonal rabbit anti-human Hb (DAKO cat no A0118) at 4.6 μg/ml in PBS and allowed to incubate overnight at 4° C. Plates were washed 5× with PBS containing 0.5% Tween-20 and then blocked with PBS containing 10% fetal calf serum for 1 hour while shaking at room temperature. 100 μl of immunoaffinity purified HDL was then added to each well (all patients were done in duplicate). In addition a 1:10 dilution of the HDL sample was prepared in SD buffer (described below) and assayed as well. The standards for the ELISA were prepared by creating a solution containing 1 ng of Hb in 100 ul of SD buffer (made by mixing one part 1M Tris pH 9, one part 1M glycine pH 2.5, one part FCS and 7 parts water). 7 serial dilutions of the standard were made in SD. SD was used as the blank in the ELISA. After aliquoting to the ELISA plates, standards and samples were incubated for 1 hour while shaking at room temperature, washed 5× with PBS 0.5% Tween-20 and to each well was added 100 ul horseradish peroxidase (HRP) conjugated goat anti-human Hb (ICL antibodies, cat # GHM-80P) at 2.5 μg/ml in PBS with 10% FCS. Plates were shaken for 1 hour at room temperature and then washed 5× with PBS 0.5% Tween-20. The plates were developed with TMB for 5 minutes, reaction stopped with 100 μl of 1M H2SO4 and absorbance read at 450 nm.

### Statistical Analysis

Selection of individuals for the HDL functional metrics component of this study were selected by the AIM HIGH coordinating center and all statistical analysis was performed by the AIM HIGH statistical center. Individuals performing biochemical analysis (measurements of HDL function and structure and Hp) had no access to any patient demographic information nor knowledge of the randomization status (niacin/placebo) of the study participants. Data are reported as the mean+/−SME for all measurements and Forest plots demonstrate the mean and 95% CI for specified endpoints. For analysis of distribution of Hb in HDL by Hp genotype the distribution of Hb did not follow a normal distribution and therefore medians were used to compare the groups rather than means and Wilcoxon test used to compare Hp 1-1 to Hp 2-2. Interaction terms were determined between niacin and Hp genotype on functional HDL metrics and between Hp genotype and Hb in HDL on HDL functional metrics (AIM-HIGH statistician to complete this describing the model used).

### Results

Demographics of AIM HIGH Participants in which HDL Functional Metrics was Assessed Stratified by Hp Genotype and Treatment Assignment.

Aliquots of de-identified 1140 plasma EDTA samples representing all DM participants of the AIM HIGH study were provided by the AIM HIGH coordinating center and an unambiguous Hp type was obtained on 1131 individuals with a Hp genotype prevalence of Hp 1-1 (16.1%), Hp 2-1 (48.8%) and Hp 2-2 (35.1%). 70 Hp 1-1 and 70 Hp 2-2 DM AIM HIGH participants, with half in the niacin and half in the placebo group, were randomly selected by the AIM HIGH coordinating center for analysis for HDL metrics at baseline and one year into the study. Table 2 below provides the baseline and one year on drug demographics and lipid profiles of these 140 AIM participants.

TABLE 2
Characteristics (baseline and year 1 (Y1)) of the 140 AIM HIGH DM participants
for whom HDL functional metrics was assessed (mean (SD))

| | Hp 11 niacin | | Hp 11 placebo | | Hp 22 niacin | | Hp 22 placebo | |
|---|---|---|---|---|---|---|---|---|
| | (N = 35) | | (N = 35) | | (N = 35) | | (N = 35) | |
| n | 34 | | 35 | | 34 | | 35 | |
| N, % male | 27 | (79.4%) | 29 | (82.9%) | 28 | (82.4%) | 33 | (94.3%) |
| Age | 68.56 | (6.85) | 63.86 | (8.29) | 63.09 | (7.91) | 64.91 | (6.62) |
| Chol base | 153.76 | (24.56) | 150.49 | (28.92) | 142.00 | (26.28) | 133.29 | (23.79) |
| HDL base | 33.91 | (5.80) | 34.97 | (5.81) | 33.74 | (6.00) | 33.91 | (4.68) |
| HDL Y1 | 40.94 | (11.48) | 37.35 | (8.01) | 43.29 | (10.93) | 36.23 | (7.59) |
| LDL base | 81.18 | (21.08) | 72.66 | (27.60) | 65.91 | (20.80) | 62.86 | (17.28) |
| LDL Y1 | 67.71 | (19.99) | 65.91 | (24.66) | 64.15 | (13.22) | 67.23 | (18.29) |
| TG base | 193.12 | (63.92) | 214.17 | (77.24) | 211.88 | (82.67) | 183.91 | (72.03) |
| TG Y1 | 174.97 | (99.63) | 227.91 | (149.68) | 164.50 | (101.69) | 188.89 | (112.63) |
| HbA1c base | 193.12 | (63.92) | 214.17 | (77.24) | 211.88 | (82.67) | 183.91 | (72.03) |

### Reverse Cholesterol Transport is Increased in Hp 1-1 by Niacin but not in Hp 2-2 Individuals.

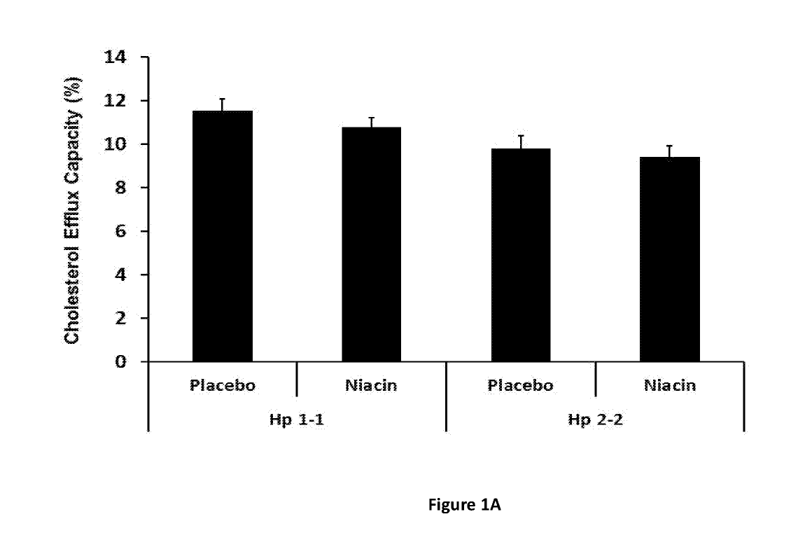

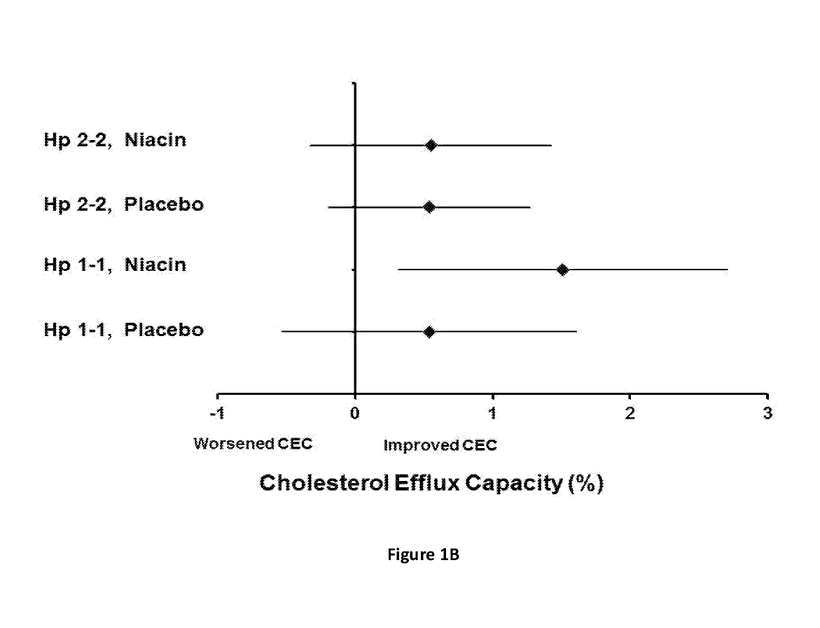

At study enrollment, cholesterol efflux capacity of serum from Hp 1-1 participants was significantly greater than that from Hp 2-2 participants (11.1+/−0.36% vs 9.6+/−0.38%, p=0.001) with the baseline cholesterol efflux capacity of the 4 study groups (Hp 1-1 and 2-2+/−niacin) shown in FIG. 1A. A significant increase in efflux capacity at one year into the study was only found in the niacin treated Hp 1-1 group (14% increase in median efflux, p<0.02 comparing efflux at baseline and after 1 year on niacin) FIG. 1B. There was no difference in efflux between Hp 2-2 individuals treated with placebo and niacin. The interaction term between Hp type and niacin on RCT function was not statistically significant.

### Total Oxidant Capacity

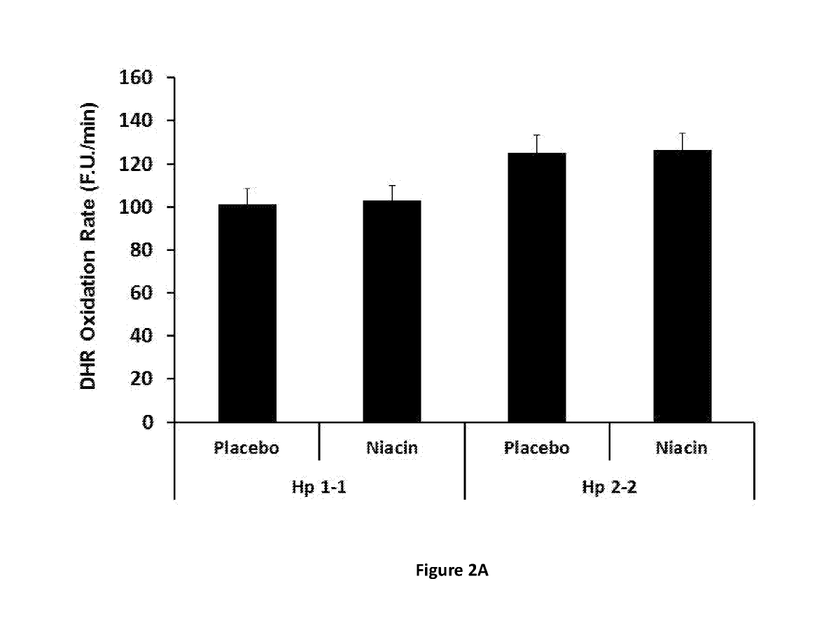

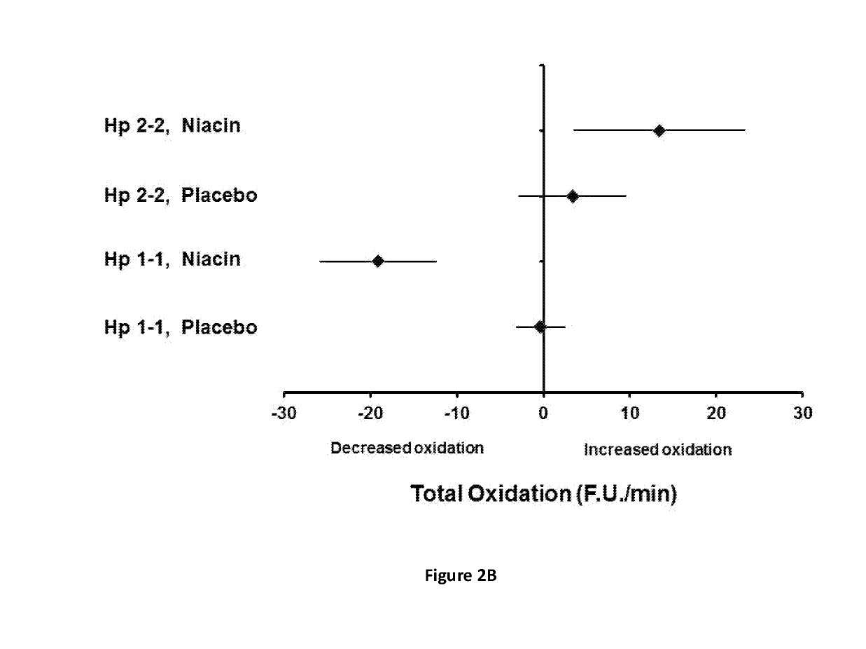

ApoB depleted serum stimulated oxidation was assessed in all 140 AIM HIGH participants at baseline and at one year of treatment as described in Methods. At study enrollment, total oxidation by ApoB depleted serum was significantly higher in Hp 2-2 vs Hp 1-1 participants with the baseline total oxidation capacity of the 4 study groups shown in FIG. 2A. Niacin treatment was associated with a significant increase in total oxidation stimulated by ApoB-depleted plasma in Hp 2-2 individuals and a significant decrease in Hp 1-1 individuals (FIG. 2B). There was a highly significant interaction between the Hp genotype and niacin on total oxidation stimulated by ApoB-depleted serum in AIM HIGH participants (p=0.001 for interaction between Hp genotype and niacin on total oxidation).

### HDL Antioxidant Capacity

The HDL mediated antioxidant capacity was determined as described in methods (FIG. 3 providing representative example of the calculation of HDL antioxidant activity on two patient's samples) on 140 AIM HIGH participants at baseline and at one year of treatment. Mean baseline antioxidant function was significantly impaired in Hp 2-2 individuals compared to Hp 1-1 individuals (Hp 2-2 participants (−1.0±3.1 antioxidant units-negative value indicating on average HDL was prooxidative in Hp 2-2) vs Hp 1-1 participants (17.5±2.5) p<0.001) with the baseline HDL antioxidant capacity of the 4 study treatment groups shown in FIG. 4A. At baseline, 42% of the Hp 2-2 AIM HIGH participants had prooxidative HDL while only 19% of the Hp 1-1 participants had prooxidative HDL (p=0.003). Niacin treatment was associated with a significant decline in the antioxidant function in Hp 2-2 individuals and an increase in HDL antioxidant function in Hp 1-1 individuals (FIG. 4B). There was a highly significant interaction between the Hp genotype and niacin on HDL antioxidant function (p<0.001 for interaction between Hp genotype and niacin treatment on the change in HDL antioxidant function).

Hb Content of HDL is Increased in Hp 2-2 and there is an Interaction Between the Hp Genotype and HDL Hb Content on HDL Function.

In a subset of the baseline AIM HIGH samples in which we measured HDL antioxidant function we have measured Hb associated with HDL by ELISA as described in the methods section. As shown in Table 3 the median amount of Hb associated with HDL in Hp 2-2 was significantly increased more than 4 fold as compared to the HDL of Hp 1-1 individuals. In a regression model with RCT as the outcome variable and Hb, Hp genotype and their interaction as the main independent variables, a significant interaction between HDL associated Hb and the Hp genotype on RCT was detected (p=0.02) with full models given in Table 4.

TABLE 3
Hb associated with HDL is Hp genotype dependent

| | Hp 1-1 (n = 20) | | Hp 2-2 (n = 20) | | p-value |
|---|---|---|---|---|---|
| Hb (median, | 0.18 | (0.12, 0.31) | 0.81 | (0.42, 1.18) | 0.0007 |
| interquartile | | | | | (Wilcoxon |
| range) | | | | | test) |
| Tertiles of | | | | | |
| Hb (%, n) | | | | | |
| <0.20 | 55 | (11) | 10 | (2) | |
| 0.20-<0.70 | 40 | (8) | 25 | (5) | |
| ≧0.70 | 5 | (1) | 65 | ((13) | 0.0002 |
| | | | | | (Chi-square) |

TABLE 4
Interaction between Hp genotype
and Hb in HDL on HDL RCT function

| Parameter | Estimate | Standard Error | t Value | p-value |
|---|---|---|---|---|
| Intercept | 11.00997269 | 2.00037233 | 5.5 | <.0001 |
| Hb | 4.63536914 | 1.53661892 | 3.02 | 0.0047 |
| Hp | 0.06210659 | 0.13512824 | 0.46 | 0.6486 |
| Hb*Hp | −0.27417624 | 0.11601088 | −2.36 | 0.0236 |

In a regression model with RCT as the outcome variable and HDL Hb and Hp genotype and their interaction as the main independent variables, a significant interaction was detected p = 0.02)

In Summary, niacin was associated with a significant increase in reverse cholesterol transport function only in individuals with the Hp 1-1 genotype. HDL antioxidant function was improved in Hp 1-1 individuals with niacin but was worsened in Hp 2-2 individuals who received niacin (p=0.0001 for interaction between Hp genotype and niacin on HDL function). In nearly half of the Hp 2-2 cohort HDL promoted rather than inhibited oxidation.

### CONCLUSIONS

There is a pharmacogenomic interaction between the Hp genotype and niacin on HDL functional metrics.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of treating a cardiovascular disease or disorder in a patient in need thereof, comprising:
a. determining the haptoglobin phenotype of a patient;
b. selecting a patient having a haptoglobin 1-1 phenotype; and
c. administering to said patient having a haptoglobin 1-1 phenotype a pharmaceutical composition comprising a therapeutically active component capable of raising HDL, thereby treating said disease or disorder.

2. The method according to claim 1, wherein the patient is a diabetic patient.

3. The method of claim 1, wherein treating said cardiovascular disease comprises at least one of preventing deterioration of said cardiovascular disease and reducing the risk for pathology resulting from said cardiovascular disease.

4. The method of claim 1, wherein said treating comprises raising the level of HDL in the blood of said patient having a haptoglobin 1-1 phenotype to at least 50 mg/dL.

5. The method of claim 1, wherein said cardiovascular disease comprises at least one of coronary atherosclerosis, dyslipidemia, type II dyslipidemia, hypercholesterolemia and myocardial infarction.

6. The method of claim 1, wherein said therapeutically active component is a CETP inhibitor.

7. The method of claim 5, wherein said CETP inhibitor is selected from the group consisting of: S-[2-({[1-(2-ethylbutyl)cyclohexyl]carbonyl}amino)phenyl] 2-methylpropanethioate, ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate, Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cyclohexanecarboxylic acid, DRL-17822 and (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one.

8. The method of claim 1, wherein said therapeutically active component is niacin.

9. The method of claim 1, wherein, the therapeutically active component capable of raising HDL is APOA1-mimetic peptide, LXR agonist, FXR agonist, Endothelial lipase inhibitor, antagonists of miR-33, ASOs targeting CETP and ApoCIII, APOA1 transcriptional upregulator, HDL mimetic prepared from recombinant APOA1, HDL mimetic manufactured from purified, authentic, human plasma APOA1 reconstituted with phospholipid, a naturally occurring mutated variant of the APOA1 protein, an oral APOA1 mimetic peptide (D-4F), recombinant human LCAT (the enzyme component of the RCT system), anti-CETP vaccine or CETP inhibitor (Cholesterylester transfer protein or fibrate.

10. The method of claim 1, wherein the pharmaceutical composition is in a form selected from the group consisting of: long acting formulation, controlled release formulation, sustained release formulation, bioadhesive formulation, mucoadhesive formulation and slow release formulation.

11. The method of claim 1, further comprising the step of assessing HDL functionality in the subject in need wherein if the HDL of the subject is dysfunctional the subject is treated with a first pharmaceutical composition comprising an antioxidant and a second pharmaceutical composition comprising a therapeutically active component capable of raising HDL.

12. A method of treating a cardiovascular disease or disorder in a patient in need thereof, comprising:
a. determining the haptoglobin phenotype of a patient;
b. selecting a patient having a haptoglobin 2-2 phenotype; and
c. administering to said patient having a haptoglobin 2-2 phenotype a first pharmaceutical composition comprising an antioxidant and a second pharmaceutical composition comprising a therapeutically active component capable of raising HDL, thereby treating said disease or disorder.

13. The method according to claim 12, wherein the patient is a diabetic patient.

14. The method of claim 12, wherein treating said cardiovascular disease comprises at least one of preventing deterioration of said cardiovascular disease and reducing the risk for said cardiovascular disease.

15. The method of claim 12, wherein said treating comprises raising the level of HDL in the blood of said patient having a haptoglobin 2-2 phenotype to at least 50 mg/dL.

16. The method of claim 12, wherein said cardiovascular disease comprises at least one of coronary atherosclerosis, dyslipidemia, type II dyslipidemia, hypercholesterolemia and myocardial infarction.

17. The method of claim 12, wherein said therapeutically active component is a CETP inhibitor.

18. The method of claim 17, wherein said CETP inhibitor is selected from the group consisting of: S-[2-({[1-(2-ethylbutyl)cyclohexyl]carbonyl}amino)phenyl] 2-methylpropanethioate, ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate, Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cyclohexanecarboxylic acid and (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one.

19. The method of claim 12, wherein said therapeutically active component is niacin.

20. The method of claim 12 The method of claim 1, wherein, the therapeutically active component capable of raising HDL is APOA1-mimetic peptide, LXR agonist, FXR agonist, Endothelial lipase inhibitor, antagonists of miR-33, ASOs targeting CETP and ApoCIII, APOA1 transcriptional upregulator, HDL mimetic prepared from recombinant APOA1, HDL mimetic manufactured from purified, authentic, human plasma APOA1 reconstituted with phospholipid, a naturally occurring mutated variant of the APOA1 protein, an oral APOA1 mimetic peptide (D-4F), recombinant human LCAT (the enzyme component of the RCT system), anti-CETP vaccine or CETP inhibitor (Cholesterylester transfer protein) or fibrate.

21. The method of claim 12, wherein said antioxidant is a tocopherol.

22. The method of claim 21, wherein the tocopherol is selected from the group consisting of alpha-tocopherol, d-alpha-tocopherol, beta-tocopherol, gamma-tocopherol and tocotrienol.

23. The method of claim 12, wherein said first pharmaceutical composition is administered prior to said second pharmaceutical composition.

24. The method of claim 12, wherein said first pharmaceutical composition and said second pharmaceutical composition are administered simultaneously.

25. The method of claim 12, further comprising the step of assessing HDL functionality in the subject in need wherein if the HDL of the subject is functional the subject is treated with a pharmaceutical composition comprising a therapeutically active component capable of raising HDL.

26. A pharmaceutical composition comprising a therapeutically active component capable of raising HDL for the treatment of a cardiovascular disease or disorder in a patient having a haptoglobin 1-1 phenotype or a patient having haptoglobin 2-2 phenotype which HDL was identified as functional.

27. A first pharmaceutical composition comprising an antioxidant and a second pharmaceutical composition comprising a therapeutically active component capable of raising HDL for the treatment of a cardiovascular disease or disorder in a patient having a haptoglobin 2-2 phenotype or a patient having haptoglobin 1-1 phenotype which HDL was identified as dysfunctional.

28. Use of pharmaceutical composition comprising a therapeutically active component capable of raising HDL for the treatment of a cardiovascular disease or disorder in a patient having a haptoglobin 1-1 phenotype or patient having haptoglobin 2-2 phenotype which HDL was identified as functional.

29. Use of a first pharmaceutical composition comprising an antioxidant, and a second pharmaceutical composition comprising a therapeutically active component capable of raising HDL for the treatment of a cardiovascular disease or disorder in a patient having a haptoglobin 2-2 phenotype or a patient having haptoglobin 1-1 phenotype which HDL was identified as dysfunctional.

30. A kit for treating a cardiovascular disease or disorder in a patient in need thereof, comprising means for determining the haptoglobin phenotype of a patient, a pharmaceutical composition comprising a therapeutically active component capable of raising HDL, and instructions for use, wherein said pharmaceutical composition is for treating a patient identified as having a haptoglobin 1-1 phenotype or patient having haptoglobin 2-2 phenotype which HDL was identified as functional.

31. A kit for treating a cardiovascular disease or disorder in a patient in need thereof, comprising means for determining the haptoglobin phenotype of a patient, a first pharmaceutical composition comprising an antioxidant, a second pharmaceutical composition comprising a therapeutically active component capable of raising HDL, and instructions for use, wherein said first and second pharmaceutical compositions are for treating a patient identified as having a haptoglobin 2-2 phenotype or a patient having haptoglobin 1-1 phenotype which HDL was identified as dysfunctional.

32. A method of determining the functionality of HDL in a subject comprising the steps of:
treating a serum or a plasma sample obtained from a subject to obtain apo-B depleted serum sample;
adding oxidation-sensitive agent;
calculating total oxidation of the oxidation-sensitive agent;
depleting HDL from the depleted apo-B serum or plasma sample by immunoprecipitation;
calculating the difference between the oxidation of the oxidation-sensitive agent slope after HDL depletion and the total oxidation slope of the oxidation-sensitive agent before HDL depletion;
wherein positive values for the difference indicate that HDL is functional in that sample as an antioxidant and negative values for the difference indicate that HDL is functional in that sample as a pro-oxidant.

33. The method of claim 32, wherein the treating of the serum or the plasma sample to obtain apo-B depleted serum sample is by treating a serum sample obtained from a subject with polyethylene glycol (PEG).

34. The method of claim 32, wherein the oxidation-sensitive agent is dihydrorhodamine.

35. The method of claim 32, wherein total oxidation is calculated by determining the rate of DHR oxidation (fluorescent units (FU)/min) after subtracting the rate of DHR oxidation observed using the same conditions but in the absence of serum.

36. The method of claim 32, wherein the immunoprecipitation is performed by using anti-human apoA1 antibody and Sepharose.

37. The method of claim 36, wherein the Sepharose is protein A/G Sepharose.

38. A kit for determining the functionality of HDL in a subject comprising: oxidation-sensitive agent; means for depleting apo-B serum from a serum sample; means for immunoprecipitation of HDL and a leaflet explaining the steps of the method for determining the functionality of HDL in a subject.

39. The kit of claim 38, wherein the means for obtaining apo-B depleted serum sample is polyethylene glycol (PEG).

40. The kit of claim 38, wherein the oxidation-sensitive agent is dihydrorhodamine—(DHR).

41. The kit of claim 38, wherein the means for immunoprecipitation is anti-human apoA1 antibody and Sepharose.

42. The kit of claim 38, wherein the Sepharose is protein A/G Sepharose.
